(19) United States
(12) Patent Application Publication
(10) Pub. No.: US 2004/0175769 A1
(43) Pub. Date: Sep. 9, 2004

(54) Assay method for hyaluronic acid

(76) Inventors: Taizo Hara, Hyogo (JP); Kenji Nakamura, Hyogo (JP)

Correspondence Address: FOLEY AND LARDNER, SUITE 500, 3000 K STREET NW, WASHINGTON, DC, 20007, US

(21) Appl. No.: 10/480,590

(22) PCT Filed: Jun. 6, 2002
(86) PCT No.: PCT/JP02/05629

(30) Foreign Application Priority Data
Jun. 12, 2001 (JP) 2001-177275

(51) Int. Cl. G01N 33/53

(52) U.S. Cl. 435/7.9

(57) ABSTRACT

The present invention provides a method for assaying hyaluronic acid more precisely and more simply than the conventional method, and relates to (1) an assay method for hyaluronic acid comprising contacting a reagent containing hyaluronic acid binding protein modified with a labeled substance with a sample containing hyaluronic acid to form a complex of the hyaluronic acid and the labeled hyaluronic acid binding protein, separating the complex and the free labeled hyaluronic acid binding protein, and measuring the labeled substance in the complex or the labeled substance in the free labeled hyaluronic acid binding protein, and (2) a reagent for an assay of hyaluronic acid comprising a labeled hyaluronic acid binding protein, which is prepared by binding a labeled substance and a hyaluronic acid binding protein through an antibody to the hyaluronic acid binding protein.

### TECHNICAL FIELD

The present invention relates to a simple and highly precise assay method for hyaluronic acid and a reagent therefor.

### BACKGROUND ART

Hyaluronic acid is a substance mainly contained in synovial fluid, vitreous humor of eyes, umbilical cord and connective tissue such as dermal surface layer of animals. Blood level of hyaluronic acid has been known to increase in a disease state such as rheumatoid arthritis, cancer and liver disease, and therefore hyaluronic acid is said to be useful for diagnosis of these diseases.

With regard to an assay method for hyaluronic acid, a sandwich method, in which a hyaluronic acid binding protein is immobilized to a solid phase, is generally known (JP-B-06-41952 and JP No.2732718). This assay method has, however, a number of disadvantages such as: (1) a hyaluronic acid binding protein as a component of the reagent, is difficult to immobilize quantitatively to a solid phase (an insoluble carrier) with a good reproducibility; (2) a calibration curve obtained by the assay is a multipoint calibration and becomes a curved line to make the assay inaccurate; (3) the system lacks simplicity due to a composition of a plurality of reagents; and (4) application to an autoanalyzer is difficult. Thus, further improvement of the assay method is attempted.

For example, in JP-A-11-14628, hyaluronic acid is assayed by carrying a hyaluronic acid binding protein on a carrier particle, reacting said protein carrying carrier with hyaluronic acid, and assaying hyaluronic acid by measuring a change of absorbance of the reaction mixture. In this method, however, disadvantageous problems are still remained. For example, it is difficult to carry a constant amount of hyaluronic acid binding protein on a carrier particle with good reproducibility.

In addition, in JP-A-2000-97940, in order to improve sensitivity, hyaluronic acid covalently bound to a protein is used in the immobilization of hyaluronic acid to a solid phase. Since this method is, however, a competitive assay method, it cannot be said that the sensitivity is dramatically improved as compared with the conventional sandwich method.

Although assay method for hyaluronic acid has been improved in various points as described hereinabove, the problem described hereinabove has not been solved, and development of a simple and highly precise assay method, which can overcome the various problems described hereinabove, has been desired.

An object of the present invention is to provide a method of simple and highly precise assay for hyaluronic acid.

### DISCLOSURE OF THE INVENTION

The present invention has been made to solve the problems described hereinabove, and relates to: “an assay method for hyaluronic acid comprising contacting a reagent containing a hyaluronic acid binding protein modified with a labeled substance with a sample containing hyaluronic acid to form a complex of hyaluronic acid and the labeled hyaluronic acid binding protein, separating the complex and the free labeled hyaluronic acid binding protein, and measuring the labeled substance in the complex or the labeled substance in the free labeled hyaluronic acid binding protein”; “a reagent for an assay of hyaluronic acid comprising a labeled hyaluronic acid binding protein, wherein a labeled substance is bound to a hyaluronic acid binding protein through an antibody to the hyaluronic acid binding protein”; and “a kit for an assay of hyaluronic acid comprising a reagent containing a labeled hyaluronic acid binding protein and a standard hyaluronic acid, wherein a labeled substance is bound to a hyaluronic acid binding protein through an antibody to the hyaluronic acid binding protein.”

The inventors of the present invention have studied extensively for a more accurate and simpler assay method for hyaluronic acid, and found that hyaluronic acid in a sample can be assayed highly precisely and simply with good reproducibility by reacting the sample containing hyaluronic acid with a reagent solution containing a labeled hyaluronic acid binding protein (hereinafter, abbreviated as a labeled HA binding protein) in a free state without being immobilized to a solid phase to form a complex of hyaluronic acid and the labeled HA binding protein, separating the complex and the free labeled HA binding protein by a separation and assay method other than the B/F separation method using a solid phase (insoluble carrier) immobilized with the hyaluronic acid binding protein, in other words, separating the free labeled HA binding protein and the complex without using such a solid phase, and measuring a quantity of the labeled substance in the complex or the labeled substance in the free labeled HA binding protein to assay hyaluronic acid in the sample, and thus completed the present invention.

### BRIEF DESCRIPTION OF THE DRAWINGS

FIG. 1 shows a calibration curve of an increased intensity of fluorescence of the reagent (reaction rate) and a concentration of hyaluronic acid obtained in Example 1.

FIG. 2 shows a calibration curve of an absorbance and a concentration of hyaluronic acid obtained in Comparative Example 1.

FIG. 3 shows a correlation between a hyaluronic acid concentration in a sample calculated by the method of the present invention (Example 1) and a hyaluronic acid concentration obtained by the conventional sandwich method (Comparative Example 1).

### BEST MODE FOR CARRYING OUT THE INVENTION

The hyaluronic acid binding protein (hereinafter, abbreviated as a HA binding protein) in the present invention is not particularly limited, so long as it is a protein having a hyaluronic acid binding domain in the protein selected from a group consisting of proteoglycan, link protein, hyaluronectin and the like. The protein may be the above exemplified protein itself, a part of the protein containing the hyaluronic acid binding domain in the above protein or a substance containing the part of the protein, and a gene recombinant protein, in which the hyaluronic acid binding domain in the above protein is excised and integrated into the other protein.

Method for labeling a HA binding protein may be a method generally used in this field, and preferably a method for binding a HA binding protein and a labeled substance through a substance having an affinity to the HA binding protein (hereinafter, abbreviated as an affinity substance to HA binding protein).

The affinity substance to HA binding protein of the present invention may be a substance having an affinity to a Ha binding protein, and includes an antibody to a Ha binding protein, preferably, a monoclonal antibody. When an antibody is used as an affinity substance to HA binding protein, the antibody is appropriately digested with an enzyme such as pepsin and papain, and is preferably used as Fab, Fab′ or (Fab′)², more preferably as Fab or Fab′ which is bound to the HA binding protein at a ratio of 1:1. When a polyclonal antibody is used as an affinity substance to HA binding protein, said antibody can be prepared by the conventional method, for example, by immunizing an animal such as equine, bovine, sheep, rabbit, goat, rat and mouse with the HA binding protein according to the method described in “Introduction to Experimental Immunology, 2nd printing, Matsuhashi, N. et al., K.K. Gakkai Publ. Center, 1981”. When a monoclonal antibody is used as an affinity substance to HA binding protein, said antibody can be prepared by the conventional method, namely, produced by a hybridoma obtained by fusing cells from mouse tumor cell line and spleen cells of mouse previously immunized with the HA binding protein, according to the cell fusion method established by KÖhler and Milstein (G. KÖhler and C. Milstein, Nature, 256, 495, 1975).

The labeled substance in the present invention includes, for example, enzymes such as alkaline phosphatase (ALP), β-galactosidase (β-Gal), peroxidase (POD), microperoxidase, glucoseoxidase (GOD), glucose-6-phosphate dehydrogenase (G6PDH), malate dehydrogenase and luciferase; pigments such as Coomassie Brilliant Blue R250 and methyl orange; radioisotopes such as ^{99m}Tc, ¹³¹I, ¹²⁵I, ¹⁴C, ³H, ³²P and ³⁵S; fluorescent substances such as fluorescein, rhodamine, dansyl, fluorescamine, coumarin, naphthylamine, or derivatives thereof, fluorescent rare earth pigments [for example, a substance consisting of a combination of a rare earth metal such as samarium (Sm), europium (Eu), terbium (Tb) or dysprosium (Dy) and a chelate compound such as 4,4′-bis (1″,1″,1″,2″,2″,3″,3″-heptafluoro-4″, 6″-hexanedion-6″-yl) chlorosulfo-o-terphenyl (BHHCT), 4,7-bis (chlorosulfonyl)-1,10-phenanthroline-2,9-dicarboxylic acid (BCPDA), and β-naphthyltrifluoroacetic acid], and nucleic acid binding fluorescent pigments; luminescent substances such as luciferin, isoluminol, luminol and bis(2,4,6-trifluorophenyl)oxalate; substances having an absorption in the ultraviolet region such as phenol, naphthol, anthracene, or derivatives thereof; and substances having a property as a spin labeling agent represented by a compound having an oxyl group such as 4-amino-2,2,6,6-tetramethylpyperidine-1-oxyl, 3-amino-2,2,5,5-tetramethylpyrrolidine-1-oxyl, 2,6-di-t-butyl-α-(3,5-di-t-butyl-4-oxo-2,5-cyclohexadiene-1-ylidene)-p-tolyloxyl (hereinafter, the above substances are sometimes abbreviated as major labeled substances).

The nucleic acid binding fluorescent pigment hereinabove means a substance emitting strong fluorescence by binding to a nucleic acid strand. The nucleic acid binding fluorescent pigment includes, for example, a substance inserted to between bases of a nucleic acid sequence, i.e. intercalating pigments [acridine pigments such as acridine orange; ethidium compounds such as ethidium bromide, ethidium homodimer 1 (EthD-1), ethidium homodimer 2 (EthD-2), ethidium bromide monoazide (EMA) and dihydroethidium; iodine compounds such as iodinated propydium and iodinated hexydium; 7-aminoactinomycin D (7-AAD); cyanine dimer type pigments such as POPO-1, BOBO-1, YOYO-1, TOTO-1, JOJO-1, POPO-3, LOLO-1, BOBO-3, YOYO-3 and TOTO-3 (all trade names of products from Molecular Probe Inc.); cyanine monomer type pigments such as PO-PRO-1, BO-PRO-1, YO-PRO-1, TO-PRO-1, JO-PRO-1, PO-PRO-3, LO-PRO-1, BO-PRO-3, YO-PRO-3, TO-PRO-3 and TO-PRO-5 (all trade names of products from Molecular Probe Inc.); SYTOX type of pigments such as SYBR Gold, SYBR Green I and SYBR Green II, SYTOX Green, SYTOX Blue and SYTOX Orange (all trade names of products from Molecular Probe Inc.)]; substances to bind to a minor group in DNA double helix [for example, 4,6-diamidino-2-phenylindol (DAPI: trade name of a product from Molecular Probe Inc.), pentahydrate (bis-benzimide) (Hoechst 33258: trade name of a product from Molecular Probe Inc.), trihydrochloride (Hoechst 33342: trade name of a product from Molecular Probe Inc.), and bisbenzimide pigment (Hoechst 34580: trade name of a product from Molecular Probe Inc.)]; and substances to specifically bind to an adenine-thymine (A-T) sequence [for example, acridine pigments such as 9-amino-6-chloro-2-methoxyacridine (ACMA) and bis-(6-chloro-2-methoxy-9-acridinyl)spermine (acridine homodimer), and substances such as hydroxystilbamidine].

Among the above labeled substances of the present invention, preferable examples are enzymes such as alkaline phosphatase (ALP), β-galactosidase (β-Gal), peroxidase (POD), microperoxidase, glucoseoxidase (GOD), glucose-6-phosphate dehydrogenase (G6PDH), malate dehydrogenase and luciferase, and more preferable examples are peroxidase (POD) and the like.

In addition, a substance prepared by binding the major labeled substance and a substance which can bind to the affinity substance to HA binding protein (hereinafter, this type of substances are sometimes abbreviated as quasi-labeled substances) may be included in the labeled substance of the present invention. For example, a nucleic acid strand bound with the major labeled substance hereinabove and avidin (or streptavidin) or biotin bound with the major labeled substance may be included within the labeled substance of the present invention. Preparation of such a quasi-labeled substance can be performed, for example, according to a known chemical binding method using, for example, a crosslinking agent (e.g. the method described in Anal. Biochem. 223, 142-148, 1994).

The nucleic acid strand, which can be used in the quasi-labeled substance hereinabove, has a fundamental unit of nucleotide residue consisting of purine base or pyrimidine base, pentose as a sugar moiety, and phosphate, and is a polymerized stranded polynucleotide in which each nucleotide is linked to between carbons at position-3′ and 5′ of the sugar by diester linkage through the phosphate. The nucleic acid strand include a RNA having ribose as a sugar moiety or/and a DNA having deoxyribose as a sugar moiety. Further, said nucleic acid strand may be either of a single strand or multiple strands consisting of two or more nucleic acid strands. The nucleic acid strand used in the present invention can be prepared by known methods such as a chemical synthetic method, a method of extracting and purifying from cells derived from microorganisms, insects, animals and plants, a method comprising culturing the cells described hereinabove, to which a proper vector gene such as plasmid, phage or cosmid is transferred, and extracting and purifying the proliferated vector by cell culture or the like, and a method utilizing a gene amplification technique such as PCR (refer to, for example, “Molecular Cloning, A Laboratory Manual”, 2nd Ed., J. Sambrook et al., Cold Spring Harbor Laboratory Press). The nucleic acid strand obtained by such method may be prepared in a desired length by appropriately purifying after cleaving by chemical degradation or a nuclease such as a restriction enzyme. Further, such nucleic acid strand may be suitably modified with a proper substance, and the modification can be performed according to the known methods.

Length of the nucleic acid strand used is normally 1 bp to 1000 kbp, preferably 5 bp to 100 kbp, and more preferably 10 bp to 50 kbp.

In the present invention, a method for binding the nucleic acid strand and the major labeled substance includes a method similar to the method for preparing the quasi-labeled substance hereinabove, and binding of the nucleic acid strand and the nucleic acid binding fluorescent pigment can be performed as follows.

Namely, the binding may be performed by contacting a nucleic acid strand and a nucleic acid binding fluorescent pigment at an adequate temperature for an appropriate time in water or a buffer solution such as Tris buffer, phosphate buffer, veronal buffer, borate buffer, Good's buffer, SSC buffer, TBE buffer and TAE buffer, which are commonly used in the hybridization technique and immunological experiment, according to the conventional method (for example, the method described in “Handbook of Fluorescent Probe and Research Chemicals, 7th Ed., Chap. 8, Molecular Probe Inc.).

In the above method, contact of the nucleic acid and the nucleic acid binding fluorescent pigment may be performed by adding the nucleic acid and the nucleic acid binding fluorescent pigment directly to water or the buffer described hereinabove, dissolving, dispersing or suspending, and admixing and contacting with each other, or by adding each of the nucleic acid and the nucleic acid binding fluorescent pigment to water or the buffer described hereinabove, dissolving, dispersing or suspending to prepare a liquid state mixture in advance, then admixing and contacting with each other.

When a nucleic acid strand bound with a nucleic acid binding fluorescent pigment is used as a quasi-labeled substance in the present invention, a timing for binding each other is not particularly limited, and may be before, simultaneously with or after formation of a complex of the HA binding protein, the affinity substance to HA binding protein, the nucleic acid strand and the nucleic acid binding fluorescent pigment (hereinafter, the complex is sometimes designated as a HA binding protein—affinity substance to HA binding protein—nucleic acid strand—nucleic acid binding fluorescent pigment), or a complex of a HA binding protein—a nucleic acid strand—a nucleic acid binding fluorescent pigment.

When avidin (or streptavidin) or biotin bound with the major labeled substance is used as a quasi-labeled substance, the desired labeled HA binding protein can be obtained by reacting the quasi-labeled substance with the affinity substance to HA binding protein which is bound with biotin or avidin (or streptavidin), then reacting with the HA binding protein, or by reacting the quasi-labeled substance with the HA binding protein which is bound with biotin or avidin (or streptavidin).

The HA binding protein, which is modified with the labeled substance, in the present invention can be obtained generally by reacting and binding three substances of the HA binding protein, the affinity substance to HA binding protein and the labeled substance as described hereinabove. It is preferable to bind the labeled substance with the affinity substance to HA binding protein, and further to bind with the HA binding protein. In this case, molar ratio of the HA binding protein and the labeled substance is preferably 1:1. By setting the molar ratio being 1:1, a molar quantity of the labeled substance bound with hyaluronic acid can be maintained as constant, then hyaluronic acid can be assayed with good reproducibility and high precision.

Further, the substance, which is obtained by reacting and binding the HA binding protein and the labeled substance without linking through the affinity substance to HA binding protein, can be included within the labeled HA binding protein of the present invention. The labeled substance used herein may be either the major labeled substance or the quasi-labeled substance, and when the labeled HA binding protein is prepared using such labeled substance, a final molar ratio of the HA binding protein and the labeled substance is preferably set to 1:1.

Preparative methods of the labeled HA binding protein in the present invention are concretely shown below including (1) a method for binding the labeled substance and the HA binding protein through the affinity substance of HA binding protein, (2) a method for directly binding the labeled substance and the HA binding protein, and (3) a method for binding the labeled substance and the HA binding protein when the nucleic acid strand bound with the major labeled substance is used as a labeled substance.

(1) Method for Binding the Labeled Substance and the HA Binding Protein Through the Affinity Substance of HA Binding Protein

Method for modifying the affinity substance of HA binding protein with the labeled substance described hereinabove can be performed by binding, directly or through a linker, functional groups respectively in the labeled substance and the affinity substance to HA binding protein. Binding method includes any of the known conventional labeling methods generally used in the known techniques such as EIA, RIA or FIA without exception (e.g. refer to the descriptions in “Experimental Medicinal Chemistry”, Vol. 8, Ed. by Yamamura, Yuichi, 1st Ed., Nakayama Publ., 1971; “Illustrated Fluorescent Antibody”, Kawau, Akira, 1st Ed., K.K. Soft Science Inc., 1983; “Enzyme Immunoassay”, Ed. by Ishikawa, Eiji et al., 3rd Ed., Igaku Shoin Publ., 1987; and “Molecular Cloning, A Laboratory Manual”, 2nd Ed., J. Sambrook et al., Cold Spring Harbor Laboratory Press), and the method can be performed according to the method hereinabove. Among the above methods, a method which can bind the affinity substance to HA binding protein and the labeled substance in a ratio of 1:1 is preferable. An example of this method is a preparative method using Fab′ in an anti-HA binding protein monoclonal antibody, and using, for example, commercially available succinimidyl 4-[p-maleimidophenyl] butyrate (SMPB, made by PIERCE Inc.) as a crosslinking agent so as to bind one labeled substance to a SH group thereof. Because, by binding the thus obtained labeled affinity substance to HA binding protein and the HA binding protein, a labeled HA binding protein, in which the labeled substance and the HA binding protein are bound in a ratio of 1:1, can be easily obtained. Binding of the labeled affinity substance to HA binding protein and the HA binding protein can be performed, for example, when an anti-HA binding protein monoclonal antibody is used as a labeled affinity substance to HA binding protein, by reacting the labeled affinity substance to HA binding protein and the HA binding protein according to the reaction conditions for performing a known antigen-antibody reaction generally performed in the known techniques such as EIA, RIA or FIA.

(2) Method for Directly Binding the Labeled Substance and the HA Binding Protein

Method for directly binding the labeled substance and the HA binding protein as described herinabove can be performed by binding, directly or through a linker, functional groups in the labeled substance and the HA binding protein. The binding method may include the conventional methods generally used in this field such as the known conventional labeling methods generally performed in the techniques such as EIA, RIA, FIA or the hybridization method (e.g. refer to the descriptions in “Experimental Medicinal Chemistry”, Vol. 8, Ed. by Yamamura, Yuichi, 1st Ed., Nakayama Publ., 1971; “Illustrated Fluorescent Antibody”, Kawau, Akira, 1st Ed., K.K. Soft Science Inc., 1983; “Enzyme Immunoassay”, Ed. by Ishikawa, Eiji et al., 3rd Ed., Igaku Shoin Publ., 1987; and “Molecular Cloning A Laboratory Manual”, 2nd Ed., J. Sambrook et al., Cold Spring Harbor Laboratory Press), and the conventional method utilizing a reaction of avidin (streptavidin) and biotin. Among the methods hereinabove, for example, a method for binding an amino group in the HA binding protein with a functional group, which can bind to the amino group, in the labeled substance is preferable, and the method for binding the labeled substance with the HA binding protein in a ratio of 1:1 is more preferable.

(3) Method for Binding the Labeled Substance and the HA Binding Protein when the Nucleic Acid Strand Bound with the Major Labeled Substance is Used as a Labeled Substance

When the nucleic acid strand bound with the major labeled substance is used as a labeled substance, either of (1) a method for binding the labeled substance and the HA binding protein through an affinity substance to HA binding protein, or (2) a method for directly binding the labeled substance and the HA binding protein, may be used, and preparation thereof can be performed according to the methods described hereinabove.

Here, a method for binding the labeled substance and the HA binding protein when the nucleic acid strand is used may be performed, after previously introducing a reactive functional group into the nucleic acid strand, by binding the HA binding protein or the affinity substance to HA binding protein and the nucleic acid strand introduced with the reactive functional group by the binding method described hereinabove. As a method for introducing the reactive functional group into the nucleic acid strand, such known methods may be used as: a method for introducing the reactive functional group by bonding a compound having the reactive functional group to 5′-triphosphate group in a terminal of the nucleic acid (e.g. a compound having an amino group such as N-trifluoroacetylamino-alkylamine, a compound having a thiol group such as cystamine, a compound having biotin such as N-biotinylamino-alkylamine, and a compound having a maleimide group such as maleimidealkylamine) through a phosphoamidite linkage using a condensing agent such as 1-ethyl-3-(3-dimethyl-aminopropyl)carbodiimide (EDC), hydrochloride (WSC), or the like [Nucleic Acid Res. (1988) 16, 3671, Chu, B.C. et al.]; a method for introducing the reactive functional group by bonding a compound having the reactive functional group to 3′-hydroxyl group in a terminal of the nucleic acid (e.g. a compound having an amino group such as N-trifluoroacetylamino-alkyl carboxylic acid, a compound having biotin such as N-biotinylamino-alkyl carboxylic acid, and a compound having a maleimide group such as maleimidealkyl carboxylic acid) through an ester linkage using a condensing agent such as 1-ethyl-3-(3-dimethyl-aminopropyl)carbodiimide (EDC), hydrochloride (WSC), or the like, or by directly reacting the activated ester compound [Nucleic Acid Res. (1986) 14, 6115, Jabloski et al.], a method for introducing an amino-group-reactive linker into a restriction enzyme fragment having a single strand with a protruding end having a base with amino group (adenine or cytosine) by reacting said linker having said single strand with a protruding end [“Chemistry of Protein and Crosslinking”, Shan S. Wong, (1991), Published by CRC Press]; a method for incorporating a nucleotide monomer introduced with the reactive functional group into a restriction enzyme fragment having a single strand with a protruding end by using a blunting enzyme (T4DNA polymerase, DNA blunting enzyme, and the like) (“Molecular Cloning, A Laboratory Manual”, 2nd Ed., J. Sambrook et al., Cold Spring Harbor Laboratory Press); a method for constructing hybridization in a single strand with a protruding end of a restriction enzyme fragment after introducing the reactive functional group into 5′-terminal of an oligonucleotide having a sequence complementary to a sequence of the single strand region in the restriction enzyme fragment which forms a single strand with a protruding end (“Molecular Cloning A Laboratory Manual”, 2nd Ed., J. Sambrook et al., Cold Spring Harbor Laboratory Press); and a method obtaining a nucleic acid strand introduced with a reactive functional group at 5′-terminal thereof as a PCR product by performing PCR using a PCR primer introduced with the reactive functional group at 5′-terminal thereof (“Molecular Cloning A Laboratory Manual”, 2nd Ed., J. Sambrook et al., Cold Spring Harbor Laboratory Press), and the like. When the single strand nucleic acid is used, the nucleic acid strand introduced with the reactive functional group can be obtained by a method for constructing hybridization of an oligonucleotide having a sequence complementary to 5′-terminal region of the single strand nucleic acid, in 5′-terminal introduced with the reactive functional group (“Molecular Cloning A Laboratory Manual”, 2nd Ed., J. Sambrook et al., Cold Spring Harbor Laboratory Press). The reactive functional groups used in the case described hereinabove include, for example, hydroxyl, alkyl halide, isothiocyanate, avidin, biotinyl, carboxyl, ketone, maleimide, activated ester, sulfonic acid halide, carboxylic acid halide, amino, sulfate and aldehyde groups.

In the binding method hereinabove, if the nucleic acid strand has a functional group, which can be bound with the HA binding protein or the affinity substance to HA binding protein, in bilateral terminals of the sequence, it is preferable to use a method wherein a nucleic acid strand is previously enzymatically or chemically cleaved to prepare a cleaved nucleic acid strand introduced with the reactive functional group into an unilateral terminal, then the cleaved nucleic acid strand is bound with the HA binding protein or the affinity substance to HA binding protein, or a method wherein a nucleic acid strand is bound with the HA binding protein or the affinity substance to HA binding protein to prepare a nucleic acid strand introduced with the HA binding protein or the affinity substance to HA binding protein in bilateral terminals of the nucleic acid strand, then the thus obtained nucleic acid strand is enzymatically or chemically cleaved to prepare a nucleic acid strand introduced with the HA binding protein or the affinity substance to HA binding protein in an unilateral terminal of the nucleic acid strand.

Method for separating the hyaluronic acid-labeled HA binding protein complex and the free labeled HA binding protein in the present invention (hereinafter, abbreviated as the separation method of the present invention) includes any known separation and analysis method exept for the B/F separation method (sandwich method) using a solid phase (insoluble carrier layer) immobilized with the HA binding protein, in other words, any method without using such solid phase. The method includes, for example, a method using a chromatography, a high performance liquid chromatography, an electrophoresis and a capillary electrophoresis or a method using an auto-immunoassay system such as LiBASys (made by Shimadzu Corp.), preferably a method using a high performance liquid chromatography, a capillary electrophoresis or an auto-immunoassay system, and more preferably a method using an auto-immunoassay system. With regard to concrete conditions of the analysis, any condition may be used so long as the hyaluronic acid-labeled HA binding protein complex and the free labeled HA binding protein can be separated. For example, when separation is conducted by using HPLC, it can be performed according to the method described in Anal. Chem. 65, 5, 613-616 (1993) or JP-A-09-301995, and when capillary electrophoresis is used, it can be performed according to the method described in J. Chromatogr. 593, 253-258 (1992) or Anal. Chem. 64, 1926-1932 (1992). Further, when, for example, LiBASys is used as an auto-immunoassay system, it can be performed according to the method described in Bio Sample Analysis, 22, No. 4, 303-308 (1999).

Assay of hyaluronic acid in the present invention can be performed, for example, by contacting a reagent containing a free labeled HA binding protein and a sample containing hyaluronic acid both in a free state in each solution to form a hyaluronic acid-labeled HA binding protein complex, separating the complex and the free labeled HA binding protein by using the separation method hereinabove, and measuring the labeled substance in the complex or the labeled substance in the free labeled HA binding protein. Alternatively, assay of hyaluronic acid in the present invention can be performed, for example, by contacting a reagent containing a labeled affinity substance to HA binding protein and a sample containing hyaluronic acid both in a free state in each solution to form a hyaluronic acid—HA binding protein—labeled HA binding protein complex, conducting the same separation method as hereinabove, separating the labeled substance in the complex or the free labeled HA binding protein, and measuring the labeled substance.

The labeled HA binding protein used hereinabove is preferably the one in which the labeled substance and the HA binding protein are bound in a molar ratio of 1:1. Use of such labeled HA binding protein enables to assay hyaluronic acid with good reproducibility and high precision, and at the same time, minimize a variation of measuring sensitivity found among production lots of the labeled HA binding protein used.

The assay of hyaluronic acid in the present invention can be performed concretely as follows.

Namely, for example, a reagent containing a labeled HA binding protein is added to a sample containing hyaluronic acid, and the mixture is allowed to stand generally at 5 to 40° C., preferably at 5 to 15° C., generally for 3 to 60 minutes, preferably for 3 to 20 minutes, then the hyaluronic acid-labeled HA binding protein complex and the free labeled HA binding protein are separated by using the auto-immunoassay analyzer as described hereinabove, and the labeled substance in the complex or the labeled substance in the free labeled HA binding protein is assayed by an appropriate method. The labeled substance can be assayed as follows: when the labeled substance is enzyme, the assay can be performed according to the conventional assay method such as EIA and hybridization technique, for example, a method described in “Enzyme Immunoassay” in Protein, Nucleic Acid and Enzyme, Supplement No. 31, Kitagawa, T. etal. Ed., p. 51-63, Kyoritsu Publ., Published Sep. 10, 1987; when the labeled substance is radioactive material, the assay can be performed according to the conventional assay method such as RIA and hybridization technique, and using a measuring apparatus suitably selected from immersion GM counter, liquid scintillation counter and well-type scintillation counter depending on a kind and strength of radiation emitted from the radioactive material (e.g. “Experimental Medicinal Chemistry”, Vol. 8, Yamamura, Y. Ed., 1st Ed., Nakayama Publ., 1971; “Tracer Technique, Lower Vol.” in “Experimental Biochemistry 2”, Takemura, A. and Honjo, T., p. 501-525, K.K. Tokyo Kagaku Dojin, Published Feb. 25, 1977); when the labeled substance is fluorescent material, the assay can be performed according to the conventional assay method such as FIA and hybridization technique using a measuring apparatus such as a fluorescence photometer and a confocal laser microscope, for example, as described in “Illustrated Fluorescent Antibody Technique”, by Kawaoi, A., 1st Ed., K.K. Soft Science, 1983, and “Chemistry of Nucleic Acids III” in “Experimental Biochemistry 2”, Saneyoshi, M., p. 299-318, K.K. Tokyo Kagaku Dojin, Published Dec. 15, 1977; and when the labeled substance is a luminescent one, the assay can be performed according to the conventional assay method using a measuring apparatus such as photon counter, for example, as described in “Enzyme Immunoassay” in Protein, Nucleic Acid and Enzyme, Supplement No. 31, Kitagawa, T. et al. Ed., p. 252-263, Kyoritsu Publ., Published Sep. 10, 1987. Further, when the labeled substance has a property of ultraviolet absorption, the assay can be performed by the conventional method using a measuring apparatus such as spectrophotometer; when the labeled substance has a property of color development, the assay can be performed by the conventional method using a measuring apparatus such as spectrophotometer or a microscope; and when the labeled substance has a spin property, the assay can be performed by the conventional method using an electron spin resonance apparatus, for example, according to the method described in “Enzyme Immunoassay” in Protein, Nucleic Acid and Enzyme, Supplement No. 31, Kitagawa, T. et al. Ed., p. 264-271, Kyoritsu Publ., Published Sep. 10, 1987.

Concentration of the labeled HA binding protein to be used in the reaction in the assay method of the present invention is varied depending on setting of the detection limit for hyaluronic acid, and generally higher than the concentration which can be bound with total amount of hyaluronic acid corresponding to the predetermined detection limit of concentration. The concentration of the labeled HA binding protein is preferably five-fold concentration or more of the above concentration, more preferably five-fold concentration or more of the above concentration. For example, when the HA binding protein—anti-HA binding protein monoclonal antibody—POD is used as a labeled HA binding protein, a concentration thereof is generally 1×10⁻⁹ M to 1×10⁻⁶ M, preferably 5×10⁻⁹ M to 5×10⁻⁷M. When a combination of the labeled substance, the affinity substance to HA binding protein and the HA binding protein, or a combination of the labeled substance and the HA binding protein is used in place of the labeled HA binding protein, a concentration of each substance can be set so that a concentration of the labeled HA binding substance formed by a reaction thereof becomes equal to the above concentration. Further, pH in the reaction is not particularly limited so long as the formation of the complex is not inhibited, and generally ranges at pH 5 to 10, preferably pH 6 to 8. Temperature of the reaction is also not particularly limited so long as the formation of the complex is not inhibited, and generally ranges at 5 to 40° C., preferably at 5 to 15° C. Reaction time depends on the reaction conditions such as the labeled HA binding substance used, pH and temperature, and ranges from several seconds to several hours depending on each of the conditions.

Solution containing the labeled HA binding protein used in the assay of hyaluronic acid of the present invention is generally a solution of the labeled HA binding protein dissolved in a suitable buffer solution. The buffer solution used for this purpose includes any buffer generally used in the immunoassay, for example, Tris buffer, phosphate buffer, veronal buffer, borate buffer, Good's buffer and N-(2-acetamide)-2-aminoethanesulfonic acid buffer (ACES buffer), and concentration thereof is generally 5 to 300 mM, preferably 10 to 150 mM, and pH thereof ranges generally at 5 to 10, preferably at 6 to 8.

Concentration of the labeled HA binding protein in the reagent for assay of hyaluronic acid of the present invention is varied depending on a type of the labeled HA binding protein used, but concentration thereof may be set so that the concentration in the reaction becomes the concentration described hereinabove, and may be suitably selected so as to range generally in 1×10⁻⁹ M to 1×10⁻⁶ M, preferably in 5×10⁻⁹ M to 5×10⁻⁷ M.

Reagent for assay of the present invention can be a reagent containing the labeled HA binding protein, for example, a reagent which can finally form the labeled HA binding protein, such as a reagent consisting of the labeled substance and the HA binding protein, a reagent consisting of the labeled substance, the labeled affinity substance to HA binding protein and the HA binding protein, and a reagent consisting of the affinity substance to HA binding protein and the HA binding protein, and is preferably a reagent containing a substance prepared by binding the labeled substance and the HA binding protein through the affinity substance to HA binding protein, more preferably a reagent containing a substance prepared by binding the labeled substance and the HA binding protein through the anti-HA binding protein antibody at a ratio of 1:1. The antibody is preferably the monoclonal antibody as described hereinbefore, and among them, Fab, Fab′ and the like are more preferable.

Buffers used in the reagent for assay of hyaluronic acid of the present invention can be the same buffer used in the assay hereinabove, and concentration thereof can be set according to the concentration used in the assay of the present invention as described hereinabove, further, pH can also be set according to the pH used in the assay of the present invention as described hereinabove.

In the reagent for assay of hyaluronic acid used in the present invention, a surface active agent conventionally used in this field may coexist in a concentration range conventionally used in this field. Even under the coexistence of surface active agent, hyaluronic acid can be assayed with good reproducibility and simplicity according to the method of the present invention. Further, in the reagent for assay of hyaluronic acid used in the present invention, a promoter for immunoreaction (a promoter for agglutination reaction) (e.g. polyethylene glycol, polyvinyl alcohol and the like) may coexist in a concentration range conventionally used in this field. According to the method of the present invention, even in the coexistence of such reaction promoter, nonspecific turbidity, which may be generated due to denaturation of the protein component in the reagent for assay caused by some factors, can be suppressed or reduced.

A kit for assay of the present invention comprises the reagent for assay of the present invention and a standard substance. The standard substance can be a substance conventionally used in this field, and includes, for example, potassium hyaluronate (derived from comb, made by Wako Pure Chemical Industries, Ltd.) and sodium hyaluronate (derived from genus Streptococcus, made by Wako Pure Chemical Industries, Ltd.). Further, if the labeled substance per se cannot be detected alone, a reagent or the like containing a substrate, which can be measured by any procedure, may be added to the kit for assay hereinabove. For example, if the labeled substance is an enzyme, a reagent containing a substrate for measuring activity of the enzyme may be added. Such substrate can be appropriately selected from the substances generally used in this field depending on the enzyme used, and concentration in use can also be appropriately selected from the concentrations generally used in this field (e.g. “Enzyme Immunoassay” in Protein, Nucleic Acid and Enzyme, Supplement No. 31, Kitagawa, T. et al. Ed., p. 51-63, Kyoritsu Publ., Published Sep. 10, 1987).

The reagent and the kit for assay of the present invention are used for performing assay method of the present invention as described hereinabove, preferable embodiments and concrete examples of each constituent element are as described hereinabove.

The present invention will be further described with Examples, but the present invention is not limited by these Examples.

### EXAMPLES

### Example 1

(1) Preparation of an Affinity Substance to HA Binding Protein Modified with a Labeled Substance

HA binding protein (made by Seikagaku Corp.), which was obtained from bovine cartilage of the nasal septum and purified by a modified method of Laurent et al., was used.

A monoclonal antibody to the HA binding protein was prepared by the conventional method. The prepared anti-HA binding protein antibody was conventionally treated to obtain Fab′. A SH group in the thus obtained Fab′ and amino group of the horseradish peroxidase (POD) were bound conventionally using a crosslinking agent, SMPB (made by Piece Biotechnology, Inc.), to prepare Fab′-POD.

(2) Preparation of a Reagent for Assaying HA

The HA binding protein and Fab′-POD were dissolved in 50 mM ACES buffer [N-(2-acetamide)-2-aminoethanesulfonic acid buffer, pH 6.5] so that the concentrations of HA binding protein and Fab′-POD become 5×10⁻⁸ M and 2.5×10⁻⁷ M, respectively, to prepare a solution of reagent for assaying hyaluronic acid.

(3) Preparation of Sample

Serum was used as a sample.

(4) Preparation of a Standard Solution of Hyaluronic Acid

Potassium hyaluronate (made by Wako Pure Chemical Industries, Ltd.) was dissolved in 50 mM phosphate buffer (pH 7.0) so that the concentration thereof becomes 1,000 ng/ml to prepare a standard solution of hyaluronic acid. The standard solution of hyaluronic acid was diluted to 100, 200,300, 400, 500, 600, 700, 800 and 900 ng/ml, respectively, to prepare the standard solutions of hyaluronic acid for a calibration curve.

(5) Assay of Hyaluronic Acid Using LiBASys (Liquid-Phase Binding Auto-Immunoassay System)

Hyaluronic acid was assayed by using LiBASys (liquid-phase binding auto-immunoassay system, made by Shimadzu Corp.).

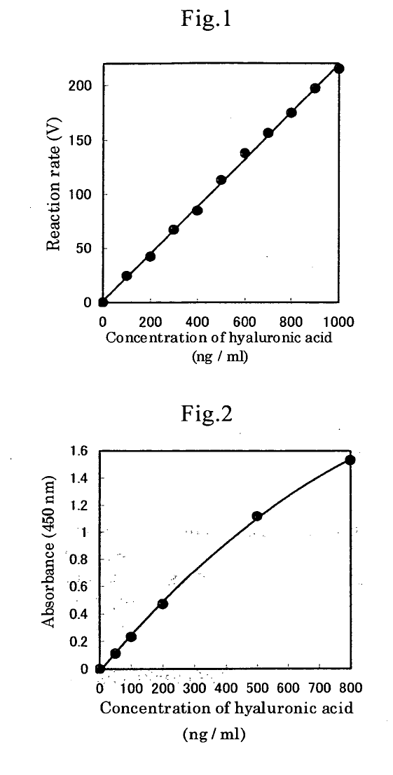

A sample or a standard solution of hyaluronic acid of 150 μl was set in a sample cup, and 10 μl thereof was collected automatically, then transferred into a reaction cuvette. Next, 100 μl of the reagent for assaying HA was automatically collected by a probe, and reacted with the sample or the standard solution of hyaluronic acid at 8° C. for 15 minutes. After completion of the reaction, 80 μl of the reaction mixture was automatically introduced into an anion exchange column by using a column probe. Free HA binding protein—anti-HA Fab′-POD was washed with 15 ml of 50 mM Tris-HCl buffer (pH 8.0) containing 0.3 M NaCl, and the complex of hyaluronic acid in the sample and HA binding protein Fab′-POD in the reagent, which was adsorbed to the anion exchange column, was eluted with 50 mM Tris-HCl buffer containing 0.9 M NaCl into a cuvette for enzyme reaction. To 1 ml of the eluent, 100 μl of a substrate solution containing 320 mM 4-acetamidophenol of a fluorescent substrate for POD and 40 mM hydrogen peroxide was automatically added, and an increase in the fluorescence generated thereby was measured in each every time. A calibration curve was prepared using known concentration of the standard hyaluronic acid depending on the increased intensity (reaction rate) of the fluorescence (FIG. 1), and a concentration of hyaluronic acid in the sample was calculated.

As apparent from the results in FIG. 1, according to the method of the present invention, a stable linear calibration curve can be obtained up to 1,000 ng/ml, and one point calibration can be made. It is understood that hyaluronic acid can be assayed with high precision by reacting in the liquid phase.

### Comparative Example 1

Hyaluronic acid in a sample was assayed using the hyaluronic acid plate “Chugai” (made by Chugai Diagnostics Science Co., Ltd.), by a conventional sandwich method using a labeled hyaluronic acid binding protein.

The sample and the standard hyaluronic acid, which were the same as in Example 1, were used.

(1) Assay of Hyaluronic Acid

The standard solution of hyaluronic acid and the sample of 50 μl each were pipetted into test tubes, respectively, and 500 μl each of the reaction buffer attached to the kit were mixed to prepare diluted solutions thereof. The reaction buffer of 100 μl for a blank solution (a concentration of hyaluronic acid is 0 ng/ml), or the diluted solution of 100 μl, was pipetted into a well of the reaction plate bound with the HA binding protein, shaken mildly, then reacted at room temperature (20 to 30° C.) for 1 hour. After completion of the reaction, the solution in each well was removed by an aspirator, and addition of 300 μl of the washing solution attached to the kit and removal of the washing solution by using aspirator were repeated for 4 times. Further, 100 μl of labeled enzyme solution (POD-labeled HA binding protein) was added to the well, shaken mildly, and reacted at room temperature (20 to 30° C.) for 30 minutes. The solution in each well was removed by an aspirator, then addition of 300 μl of the washing solution and removal of the washing solution by using aspirator were repeated for 4 times. Furthermore, 100 μl of coloring solution (3,3′,5,5′-tetramethylbenzidine and hydrogen peroxide) was added, shaken, reacted at room temperature (20 to 30° C.) for 30 minutes in the dark, then added with 100 μl of the reaction terminator attached to the kit and shook to terminate the reaction, followed by measurement of absorbance at 450 nm by using a plate reader within 30 minutes.

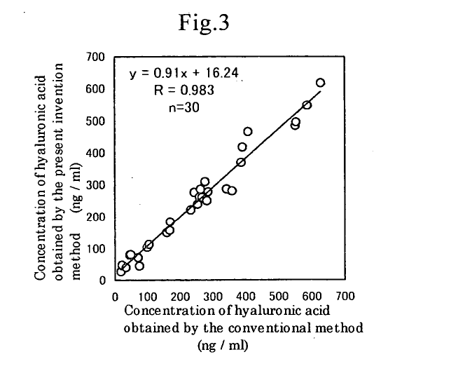

A calibration curve was prepared from the absorbance obtained by measuring the standard solutions of hyaluronic acid (FIG. 2), and a concentration of hyaluronic acid in the sample was calculated from the absorbances of the sample. A correlation between the calculated hyaluronic acid concentration in the sample and the concentration of hyaluronic acid in the sample obtained in Example 1 is shown in FIG. 3.

As apparent from the results in FIG. 3, the assay values obtained by the method of the present invention show a good correlation with those obtained by the conventional method. Consequently, from these results, it can be understood that an assay of hyaluronic acid with one-fluid reagent can be realized by the present invention, and a simple and easy hyaluronic acid assay can be performed by using an automatic analyzer (LiBASys).

### Industrial Applicability

As apparent from the description hereinabove, the present invention provides a simple and highly precise assay method for hyaluronic acid. More particularly, the present invention enables to assay hyaluronic acid with high precision and good reproducibility as well as rapidly and simply without variation in the assay values caused by a lot-to-lot variation of reagent, by using a labeled HA binding protein produced by reacting the HA binding protein and the labeled substance at a ratio of 1:1.

1. An assay method for hyaluronic acid comprising contacting a reagent containing hyaluronic acid binding protein modified with a labeled substance (a labeled hyaluronic acid binding protein) with a sample containing hyaluronic acid to form a complex of the hyaluronic acid and the labeled hyaluronic acid binding protein, separating the complex and the free labeled hyaluronic acid binding protein, and measuring the labeled substance in the complex or the labeled substance in the free labeled hyaluronic acid binding protein.

2. The assay method according to claim 1, wherein the labeled hyaluronic acid binding protein is a substance prepared by binding the labeled substance and the hyaluronic acid binding protein through a substance having an affinity to the hyaluronic acid binding protein.

3. The assay method according to claim 2, wherein the substance having an affinity to the hyaluronic acid binding protein is an antibody.

4. The assay method according to claim 3, wherein the antibody is Fab or Fab′ of anti-hyaluronic acid binding protein monoclonal antibody.

5. The assay method according to claim 4, wherein the labeled substance and the hyaluronic acid binding protein are bound at a ratio of 1:1.

6. The assay method according to claim 5, wherein the labeled substance is radioisotope, fluorescent pigment, chemiluminescent substance, spin labeling agent or enzyme, or a substance having the same.

7. The assay method according to claim 6, wherein the hyaluronic acid binding protein is a protein having the hyaluronic acid binding domain in the protein selected from a group consisting of proteoglycan, link protein and hyaluronectin.

8. The assay method according to claim 7, wherein the separation is carried out by chromatography, electrophoresis, capillary electrophoresis or a method using an automatic immunoanalyzer equipped with an ion exchange column.

9. A reagent for an assay of hyaluronic acid, comprising a labeled hyaluronic acid binding protein, which is prepared by binding a labeled substance and a hyaluronic acid binding protein through an antibody to the hyaluronic acid binding protein.

10. The reagent for the assay according to claim 9, wherein the antibody is Fab or Fab′ of anti-hyaluronic acid binding protein monoclonal antibody.

11. The reagent for the assay according to claim 10, wherein the labeled substance and the hyaluronic acid binding protein are bound at a ratio of 1:1.

12. The reagent for the assay according to claim 11, wherein the labeled substance is radioisotope, fluorescent pigment, chemiluminescent substance or enzyme.

13. The reagent for the assay according to claim 12, wherein the hyaluronic acid binding protein is a protein having a hyaluronic acid binding domain in the protein selected from a group consisting of proteoglycan, link protein and hyaluronectin.

14. A kit for an assay of hyaluronic acid, comprising a reagent containing a labeled hyaluronic acid binding protein, which is prepared by binding a labeled substance and a hyaluronic acid binding protein through an antibody to the hyaluronic acid binding protein, and a standard substance.
